(19) United States
(12) Patent Application Publication
(10) Pub. No.: US 2004/0220502 A1
(43) Pub. Date: Nov. 4, 2004

(54) Apparatus for supporting the back

(76) Inventor: Wayne R. Arden, Steamboat Springs, CO (US)

Correspondence Address: TIMOTHY J MARTIN, PC, 9250 W 5TH AVENUE, SUITE 200, LAKEWOOD, CO, 80226, US

(21) Appl. No.: 10/757,730

(22) Filed: Jan. 13, 2004

Related U.S. Application Data
(63) Continuation-in-part of application No. 09/792,527, filed on Feb. 23, 2001, now Pat. No. 6,676,619.

(51) Int. Cl. A61F 5/00

(52) U.S. Cl. 602/19

(57) ABSTRACT

A brace mounts externally of a person's torso to support the back. The brace includes a support piece that has an elongated central portion and a plurality of pairs of oppositely projecting lobe portions that extend laterally of a central portion. The lobe portions are formed of a stiff yet bendable material. A support band encircles the torso and has fasteners on its ends to releasably secure the support piece alongside the back. The support band is formed of a flexible material. The support piece can be a unitary one-piece construction with the lobe portions formed by notches. Alternatively, the lobe portions may be formed by strips of stiff yet bendable material extending transversely of the central portion. The lobe portions may be received in flexible sleeves, and the support band may include a flexible pouch to receive the support piece.

### RELATED APPLICATION

This application is a continuation-in-part of co-pending U.S. application Ser. No. 09/792,527, filed Feb. 23, 2001, now U.S. Pat. No. 6,676,619, issued Jan. 13, 2004, the contents of which are incorporated herein by reference.

### FIELD OF THE INVENTION

The present invention is broadly directed to the medical and veterinary field. More particular, the present invention concerns apparatus, such as splints, braces, temporary casts and the like. Specifically, the present invention concerns a brace which can mounted around the torso of the body alongside the back in order to support the back against injury or to relieve back pain.

### BACKGROUND OF THE INVENTION

Locomotion is a cardinal sign of animal life including humans as well as the lower animal forms. In higher living organisms, movement is typically accomplished by appendages such as legs and arms attached to a trunk or body portion of the animal. Active movement exposes the animal to various forms of trauma, particularly to the bones and joints. Such trauma can include strains, sprains, bone cracking or breaking and the like. Furthermore, natural aging processes deteriorate animal body structures with bones and joints being particularly susceptible to age-related deterioration. Even when injured or advanced in age, animals typically require some level of movement for meaningful life and often for survival.

It is known to provide auxiliary support for various body parts of both humans and domestic animals. For example, in the case of a bone crack or break, the bone may be set and a rigid cast placed around the body part corresponding to the broken or cracked bone so as to inhibit any movement during the healing process. Such casts are typically uncomfortable and cumbersome and act to severely restrict movement of the body part. As an alternative to casts, rigidifying structures in the form of rod-like or stick braces are used. Here, also, the goals are to provide auxiliary support to the body part and to severely restrict the motion of the body part as a form of protection against further injury and to promote healing. In the case of a splint, a rigidifying structure such as a rod, slate or other elongated rigid piece is placed alongside the body part, typically on each side of the joint, and mounted in position so as to restrict motion about the joint or about a fracture. The mounting of the splint is usually by a flexible wrap which, in some instances, may be an elastic support bandage or the like.

In some instances, it is sufficient to provide auxiliary support to the body part by means of a flexible resilient member such as an elastic casing, elastic wrap and the like. In the case of an elastic wrap, an elongated strip of elastic cloth is typically wound circumferentially around the body part so as to encase the body part in the mass of flexible material. Alternatively, the elastic device may be formed as a tube-like sheath that is slid onto the body part so as to encircle the body part thereby providing lateral stability. In either case, the resilient devices support the body part so as to resist undesired bending. The resilient nature of the material, while not as rigidifying as a splint or cast, has been found to supplement the body's own support structure so as to reduce pain during movement and reduce the likelihood of additional injury.

Auxiliary supports are used both as a treatment device as well as a prophylactic device. During treatment, such devices help prevent movement and promote the healing process as noted above. As a prophylactic device, such devices provide auxiliary support prior to any injury so as to reduce the likelihood of injury by augmenting the body's support framework and musculature. For example, many persons engaged in athletic endeavors will wear elastic knee braces, elbow braces, wrist braces, ankle braces and the like during the athletic activity to reduce the likelihood of injury during movement. Indeed, it is known that many athletes partially immobilize an ankle joint by adhesive taping to allow flexion of the foot with reduced risks of lateral movement that might cause a strain or sprain.

Despite the protection provided by elastic support devices, the same often provide inadequate support for the injured body part or inadequate protection against injury as a prophylactic approach. Braces and casts, on the other hand, restrict movement to a degree that prevents meaningful sufficient movement during athletic activities. Accordingly, there is a need for an apparatus that bridges the gap between braces and casts on one hand, and elastic bandages and casings on the other. There is a need for such devices that have greater support against lateral motion of the body part while still allowing sufficient flexibility of the body part for active movement.

This need is particularly true where the torso and back of a person or animal in concerned. It is known that the back is often the site of strains resulting from movement, hyperflexion or muscle pulls from lifting activities. Braces are used both as a prophylactic to help reduce or prevent injury and to support an injured back during activities. There is therefore a need for improved braces that can support the back.

### SUMMARY OF THE INVENTION

It is an object of the present invention to provide a new and useful brace adapted to support the back of a person or animal.

It is a further object of the present invention to provide a new and useful brace which may be used on injured or weakened back.

It is a further object of the present invention to provide a new and useful brace which supports the injured or weakened back while still allowing some degree motion.

It is still a further object of the present invention to provide a new and useful brace in which the support piece of the brace floats within the confines of the flexible portion of the brace to provide flexibly stiff support for an injured or weakened back.

It is still a further object of the present invention to provide an easily releasable brace as an auxiliary back support to resist injury.

It is yet another object of the present invention to provide an adjustable brace that resists impact forces and is light in weight.

A still further object of the present invention is to provide a brace that may enhance athletic performance of the wearer.

According to the present invention then, a brace is adapted to be positioned externally of a person's torso in order to support the persons back. Broadly, the brace includes a support piece that has an elongated central portion and a plurality of lobe portions extending laterally of the central portion, with these lobe portions constructed of a stiff yet bendable material. In one form of the present invention, a plurality of pairs of oppositely projecting lobe portions extend laterally of the central portion. In another embodiment of the invention, the support piece is received within a sheath that includes first and second panels of a flexible material secured together around a perimeter thereof so as to have a sheath interior. The brace optionally includes a support band that is sized and adapted to insert through the torso and is operative to releasably secure the support piece alongside the persons back when in a mounted state. The support band is constructed of a flexible material and has opposite end portions provided with cooperative fasteners whereby the end portions may be releasably secured together in a fastened state.

One embodiment of the support piece shows it being formed of a unitary one-piece construction of stiff yet bendable material. Here, the lobe portions are formed by notches located between adjacent ones of the lobe portions. While a plurality of pairs of lobe portions are contemplated, the illustrated embodiment includes three pairs of lobe portions with there being a primary pair of lobe portions and two secondary lobe portions, one on either side of the primary pair. Here, also, the primary pair of lobe portions may extend a lateral distance away from the central portion that is greater than the lobe portions of the secondary pairs. The stiff yet bendable material may be selected from a group consisting of composite materials, plastics, aramid compounds, Kevlar® (a trademark of E. I. du Pont de Nemours and Company of Wilmington, Del.), graphite, resin impregnated fabrics, other ballistic-type fibers and the like.

In another form of the invention, the central portion includes a flexible material. Here, each of the lobe portions is then formed by a strip of stiff yet bendable material secured to an extending transversely to the central portion. To this end, the strips may be received in oversized sleeves such that the strips may laterally slide therein with respect to the central portion. For example, the strips May extend perpendicularly to the central portion.

If desired, the support band can include a pouch made of a flexible material. This pouch is located medially of the end portions and has a pouch interior sized and adapted to receive the support piece. Here, again, the pouch interior is oversized relative to the support piece whereby the support piece may undergo sliding movement therein, either in the longitudinal or lateral direction, as desired. The pouch may be formed of a fabric material selected from a group consisting of substantially non-stretchy fabrics and resilient stretchy fabrics. Accordingly, the pouches and end portions may be formed of a common material and may be of an interval, one-piece construction. The pouch may also be provided with a releasable pouch closer, such as Velcro strips.

The cooperative fasteners adjacent the free ends of the end portions of the support band may be constructed so as to permit size adjustment of the band. Examples of such fasteners include hook and loop fastening strips, snaps, buttons, bayonet fasteners, to name a few.

As noted above, the support piece may optionally be received in a sheath but may be then held against the person's back by the support band. The sheath may be chosen to be of a slippery material relative to the material used for the support piece. For example, the sheath may be constructed of a material selected from a group consisting of nylon, polyester, coated (PTFE) materials and the like. Moreover, the sheath interior may have a periphery that is contoured to the shape of the support piece.

These and other objects of the present invention will become more readily appreciated and understood from a consideration of the following detailed description of the exemplary embodiments of the present invention when taken together with the accompanying drawings, in which:

### BRIEF DESCRIPTION OF THE DRAWINGS

FIG. 1 is a perspective view of a brace according to a first exemplary embodiment of the present invention with this brace being shown secured around the torso of a person alongside the person's back;

FIG. 2 is a front view in elevation showing the first exemplary embodiment of a brace according to the present invention;

FIG. 3 is a rear view in elevation showing the first exemplary embodiment of a brace according to the present invention;

FIG. 4 is a cross-sectional view taken about lines 4-4 of FIG. 2;

FIG. 5 is an exploded front view in elevation, partially broken away, showing the construction of the brace of FIGS. 2-4;

FIG. 6 is a front view in elevation showing the support piece of FIG. 5;

FIG. 7 is a front view in elevation showing the support piece of FIG. 6 received in a first exemplary embodiment of a retaining sheath;

FIG. 8 is a front view in elevation showing the support piece of FIG. 6 received in a second exemplary embodiment of a retaining sheath;

FIG. 9 is a front view in elevation, partially broken away, showing a second exemplary embodiment of the present invention; and

FIG. 10 is a front view in elevation, partially broken away, showing a third exemplary embodiment of the present invention.

### DETAILED DESCRIPTION OF THE EXEMPLARY EMBODIMENTS

The present invention is broadly directed to medical and veterinary devices, such as orthopedic devices that are used to support a body part. More particularly, the present invention is directed to a back brace which can be incorporated or used as a support device for the human back. The term “animal” includes human as well as non-human animals.

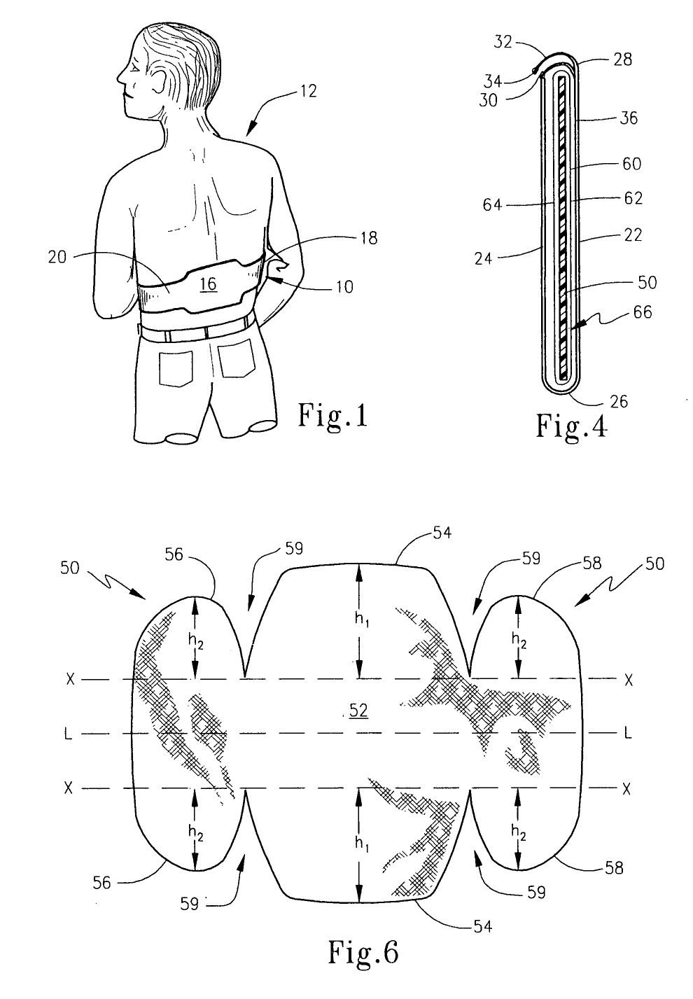

With reference first to FIG. 1, brace 10 according to a first exemplary embodiment of the present invention is shown secured around the torso of person 12 in order to support the person's back. As is shown in this figure, brace 10 includes a support band 14 that has a medially located pouch 16 from which a pair of end portions 18 and 20 oppositely project. As discussed more thoroughly below, pouch 16 is sized and adapted to receive a support piece that is held against the person's back by means of end portions 18 and 20 of support band 14. To this end, end portions 18 and 20 are provided with cooperative fasteners whereby the end portions may be releasably secured together in the fastened state.

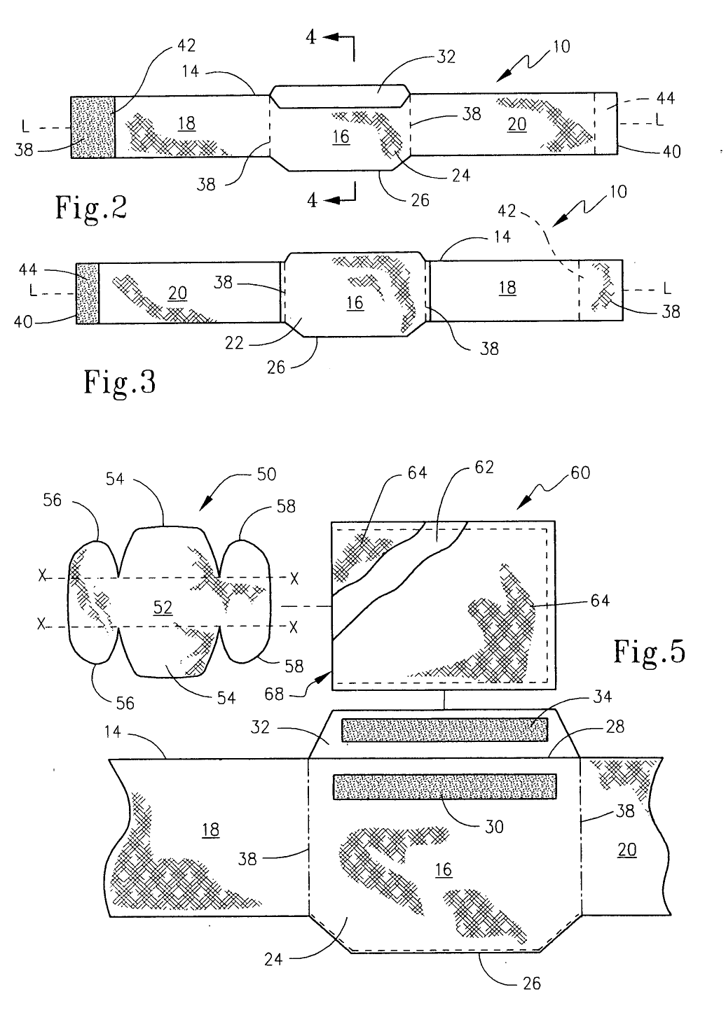

The further structure of support band 14 may be appreciated with reference to FIGS. 2-5. In these figures, it may be seen that pouch 16 is formed by a pair of panel pieces 22 and 24 that may be either of a common piece of material folded over at revers 26 or otherwise sewn together as is known in the art. An upper edge 28 of panel 24 is provided with one-half of a cooperative fastener, such as a strip 30 of loop material. Panel 22 includes a flap portion 32 that is provided with the other half of the cooperative fastener, here illustrated to be a strip of hook or filaform elements 34. This structure is also illustrated in FIG. 5, and it should be appreciated that flap 32 may be folded over so that fasteners 30 and 34 may be releasably secured together to enclose the interior 36 of pouch 16. Suitable transverse stitching 39 is provided to complete the formation of pouch 16.

End portions 18 and 20 project along a longitudinal axis “L” oppositely one another. Again, end portions 18 and 20 are provided with cooperative fasteners so that the free ends 38 and 40 thereof may be releasably secured together in a closed loop encircling the person's torso. As is shown in FIGS. 2 and 3, a panel 42 of loop material is affixed to end portion 18 adjacent to free end 38. Similarly, a panel 44 of hook or filaform elements is secured to end portion 20 adjacent to free end 40 by on the side of support band 14 opposite panel 42 of loop material. Panels 42 and 44 should be sufficiently dimensioned so as to allow some adjustability of size for differently sized torsos. Further and though not absolutely required, it is desirable that end portions 18 and 20 be constructed of a stretchy, resilient material. This allows both size adjustment and also a resilient force pressing pouch 16 against the back of the person who wears brace 10. Two examples of suitable material are nylon fabric and poly stretch fleece. In addition, while all the cooperative fasteners are illustrated to be hook and loop fasteners, it should be appreciated by the ordinarily skilled person that a wide variety of other cooperative fasteners could be employed without departing from the scope of this invention. These include fasteners such as buckle type fasteners, snaps, hook and eyes, buttons, zippers, bayonet closures, to name a few.

It is also desirable that pouch 16 be formed of flexible material. To this end, the body of support band 14 can be structured from an integral, one-piece construction of material with panel 22 and flap 32 being simply folded over with respect to panel 24 that is a co-linear joining portion for end portions 18 and 20. Alternatively, pouch 16 could be formed of a non-stretchy fabric with the stretchy resilient end portions 18 and 20 secured thereto by stitching 38. As noted above, loop fasteners fastening strip 30 and hook fastening strip 34 provides a releasable pouch closure for pouch 16.

As best illustrated in FIGS. 5 and 6, brace 10 also includes a support piece 50 that has an elongated central portion 52 located between lines “x”. Support piece 50 also includes a plurality of pairs of oppositely projecting lobe portions extending laterally of central portion 52. In the embodiment shown in FIG. 5, three such pairs are shown including a primary pair of lobe portions 54 and two pairs of secondary lobe portions 56 and 58.

Continuing with reference to FIG. 5, in the embodiment of brace 10 shown in FIGS. 1-5, support piece 50 is received in a sheath 60 formed of a flexible material. Here, a pair of panels 62 and 64 of flexible material are folded and sewn together so as to have a sheath interior 66 forming a mouth 68 through which support piece may be inserted and then the sheath support piece inserted into the interior 36 of pouch 16. To this end, it is desired that the interior of pouch 16 be oversized relative to the support piece whereby the support piece may undergo a sliding movement after being secured within interior 36 of pouch 16. Here, also, it is desired that the pouch interior be oversized in a lateral dimension so as to accommodate lateral movement in a direction transverse to axis “L”. Accordingly, for purposes herein, the word “oversized” is intended to mean sufficiently larger so as to permit sliding movement of at least 5% of the dimension of support piece 50 in the direction of such sliding movement. Further, the word “transverse” is intended to mean a crosswise direction that may be but is not necessarily perpendicular to the reference axis.

The geometry and construction of support piece 50 may be further appreciated with reference to FIG. 6. At the outset, support piece 50 is formed by a stiff yet bendable material that is suitable to provide a supportive force when held against the back. Here, support piece 50 is formed as an integral, one-piece construction of a stiff yet bendable material. The desired materials include composite materials, plastics, aramid compounds, Kevlar®, graphite, resin impregnated fabrics (which desirably would be resins that are flexible after curing) and the like. Other ballistic-type fibers are also contemplated for use in constructing the support piece. Thus, when used herein, the phase “ballistic-type material” means also conventional aramids (such as phenylenediamine terephthalamide), nylons, glass, graphite ultra high molecular weight polyethylene, polypropylene, polyvinyl alcohols and the like, whether now known or hereinafter developed.

As is seen also in FIG. 6, each of lobes 56 and 58 extend perpendicularly of central portion 52 and equidistantly thereof for a distance or height “h₂”. Similarly, each of primary lobes 54 extends perpendicularly to and equidistantly of central portion 52 for a distance or height “h₁”. In this configuration, h₁ is greater than h₂. Where support piece 50 is formed as an integral, one-piece construction, primary lobe portions 54 and secondary lobe portions 56 may be formed by notches 59 cut in the material. Finally, it may be observed with reference to FIG. 6 that each of primary lobe portions 54 extend oppositely one another in the lateral direction. Likewise, each of lobe portions 56 extend oppositely one another in the lateral direction, and each of lobe portions 58 extend oppositely one another in the lateral direction.

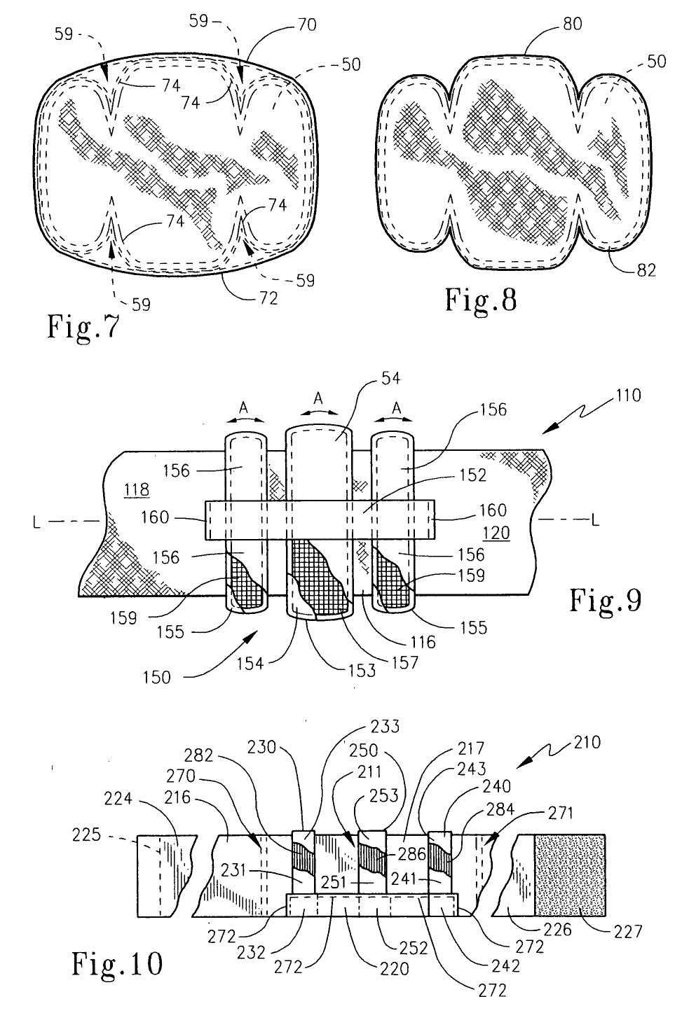

In the embodiment shown in FIGS. 1-6, support piece 50 is shown to be removably inserted in the optional sheath 60. Sheath 60, however, can be formed to entirely enclose support piece 50, such as shown in two alternative embodiments of FIGS. 7 and 8. In FIG. 7, it may be seen that support piece 50 (shown in phantom) may be received in a sheath 70 formed by two panels stitched entirely around their perimeter by stitching 72. Stitching 74 extends within notches 59 so as to lock support piece 50 therein, with sheath 70 taking a generally oval appearance.

Alternatively, as is shown in FIG. 8, support piece 50 (shown in phantom) can be received in a sheath 80 that is cut to be contoured to the perimater of support piece 50 such that the sheath interior has a periphery contoured to the shape of the support piece. Stitching 82 is provided around the peripheral margin of sheath 80 so as to contour to the shape of support piece 50. In either event, the support piece 50 received in either sheath 70 or 80 may then be inserted into pouch 16 of support band 14.

The present invention also contemplates the securing of a support piece 50, either alone or enclosed in a sheath such as sheath 60, 70 or 80, that is secured against the back by means of a traditional band, such as a weight lifter's belt, an elastic wrap, or otherwise, as is known in the art. Here, though, it is preferred that the support piece 50 be received in a sheath to increase the comfort to the wearer thereof. To this end also, the material used to form either of sheaths 60, 70 and 80 may be a slippery material relative to the construction of support piece 50. For example, the material used to form these sheaths may be nylon, polyester, coated (PTFE) and the like.

Another alternative embodiment of the present invention is shown in FIG. 9. Here, brace 110 is illustrated to include a continuous band of flexible, stretchy material so as to have end portions 118 and 120 which project oppositely a medial region 116. A support piece, generally designated as 150 includes a central portion 152 formed as an elongated strip of flexible material to which is secured, such as by sewing, adhesives, or otherwise, a plurality of sheaths including sheaths 153 and a pair of sheaths 155. Sheaths 153, 155 are secured transversely of central portion 152 and respectfully receive elongated strips 157, 159 of stiff yet bendable material of a similar construction to that of support piece 50. Accordingly, the opposite ends of strips 157 and 159 respectively form oppositely projecting lobes 154 and 156 which project oppositely one another and project transversely of central portion 152, and, specifically, perpendicularly thereto. Flexible central portion 152 is sewn to medial portion 116 by means of stitching 160 so as to secure each of Sheaths 153 and 155 thereto. This, construction, then, supports strips 157 and 159 relative to support band 110 with sheaths 153 and 155 being oversized relative to their respective strips so as to permit lateral sliding movement thereof in a direction transverse to axis “L”. Moreover, each of lobes 154 and 156 may, due to the flexibility of strip 152, pivot in the direction of arrows “A”. Finally, it may be seen that the strips are dimensioned so that, again, primary lobes 154 extend a lateral distance away from flexible central portion 152 that is greater than the lateral distance or height of lobes 156.

FIG. 10 also shows a back brace 210 that includes a support band or web 216 that is adapted to extend around the mid-section of a person's body. Support web 216 includes a medial portion 217 formed of a flexible yet substantially elastic fabric. Elastic webs 224 and 226 extend oppositely one another and are fastened to web 217 at stitching 270 and 271, respectively. Elastic webs 224 and 226 are provided with mating hook and loop fasteners such as filaform elements 225 (shown in phantom) and loop elements 227. The dimension of loop elements 227 selected so as to provide some degree of adjustment based on the attachment location of filaform elements 225 to loop elements 227 when support web 216 encircles the torso.

In any event, brace section 211 is secured to flexible web 217. Here, an attachment web 220 of brace section 211 may be sewn to flexible web 217 at stitching 272 to secure the first end portions 231, 241, and 251 of sleeves 230, 240 and 250 to flexible web 217 at junction locations 232, 242 and 252, respectively. Strips 282, 284 and 286 are received in the respective interior of sleeves 230, 240 and 250 and are sized for relative movement therein. Second end portions 233, 243 and 253 of sleeves 230, 240 and 250 are free so that sleeves 230, 240 and 250 are not attached along to web 217 along their length except at junctions 232, 242 and 252. From the above description, it should be appreciated that brace system 210 may support the lower back of a person along the long axis of the spine with sleeve 250 oriented over the spine while sleeves 230 and 240 are located parallel to the spine on opposite sides thereof.

Accordingly, the present invention has been described with some degree of particularity directed to the exemplary embodiments of the present invention. It should be appreciated, though, that the present invention is defined by the following claims construed in light of the prior art so that modifications or changes may be made to the exemplary embodiments of the present invention without departing from the inventive concepts contained herein.

I claim:

1. A brace adapted to mount externally of a person's torso in order to support the person's back, comprising:
(A) a support piece including an elongated central portion and a plurality of pairs of oppositely projecting lobe portions extending laterally of said central portion, said lobe portions constructed of a stiff yet bendable material; and
(B) a support band sized and adapted to encircle the person's torso and operative to releasably secure said support piece alongside the person's back when in a mounted state, said support band constructed of a flexible material and having opposite end portions provided with cooperative fasteners whereby said end portions may be releasably secured together in a fastened state.

2. A brace according to claim 1 wherein said central portion includes a flexible material.

3. A brace according to claim 2 wherein each of said lobe portions is formed by a strip of the stiff yet bendable material secured to and extending transversely of said central portion.

4. A brace according to claim 3 wherein said strips extend perpendicularly to said central portion.

5. A brace according to claim 3 including a plurality of sleeves each formed of a flexible material and having a sleeve interior connected to said central portion with said sleeves receiving a respective said strip therein.

6. A brace according to claim 5 wherein at least some of said sleeves are oversized such that the respective said strip may move relative to its respective said sleeve within the interior thereof.

7. A brace according to claim 5 wherein said sleeves are in parallel spaced relation to one another

8. A brace according to claim 3 wherein one of said strips is of a different width than another of said strips.

9. A brace according to claim 1 wherein the lobe portions of each said pair extend equidistantly of said central portion.

10. A brace according to claim 1 wherein said support piece is formed as a unitary one-piece construction of the stiff yet bendable material, said lobe portions being formed by notches located between adjacent ones of said lobe portions.

11. A brace according to claim 1 including at least three pairs of lobe portions, there being a primary pair of lobe portions and two secondary lobe portions, one on either side of said primary pair of lobe portions.

12. A brace according to claim 11 wherein said primary lobe portion is wider than said secondary lobe portions.

13. A brace according to claim 11 wherein the lobe portions of said primary pair extend a lateral distance away from said central portion that is greater than the lobe portions of said secondary pairs.

14. A brace according to claim 1 wherein said stiff yet bendable material is selected from a group consisting of: composite materials, plastic, aramid compounds, Kevlar®, graphite, plastic, ballistic-type materials and resin impregnated fabrics.

15. A brace according to claim 1 wherein said end portions of said support band are formed of a resiliently stretchy material.

16. A brace according to claim 1 wherein said support band includes a pouch that is formed of a flexible material and that is located medially of said end portions, said pouch having a pouch interior sizes and adapted to receive said support piece.

17. A brace according to claim 14 wherein the pouch interior is oversized relative to said support piece whereby said support piece may undergo sliding movement therein.

18. A brace according to claim 17 wherein the pouch interior is oversized in a lateral dimension.

19. A brace according to claim 14 wherein said pouch is formed of a fabric material selected from a group consisting of substantially non-stretchy fabrics and resilient stretchy fabrics.

20. A brace according to claim 19 wherein said pouch and said end portions are formed of a common material.

21. A brace according to claim 14 wherein said pouch is provided with a releasable pouch closure.

22. A brace according to claim 1 wherein said cooperative fasteners permit size adjustment of said band.

23. A brace according to claim 1 including a sheath formed of a flexible material and having a sheath interior sized and adapted to receive said support piece, said band operative to releasably secure said sheath relative to the person's body.

24. A brace according to claim 21 wherein said sheath is constructed of a material selected from a group consisting of nylon, polyester, and PTFE coated materials.

25. A brace according to claim 21 wherein the sheath interior has a periphery contoured to the shape of said support piece.

26. A brace adapted to be releasably secured externally of a person's torso in order to support the person's back, comprising:
(A) a sheath formed and including first and second panels of a flexible material secured together around a perimeter thereof so as to have a sheath interior;
(B) a support piece sized and adapted to be received in the sheath interior, said support piece including an elongated central portion defining a length for said support piece and having a longitudinally extending central axis and a plurality of pairs of oppositely projecting lobe portions extending laterally of said central portion for a respective lobe length thereby to define a width for said support piece, said lobe portions constructed of a stiff yet bendable material and each extending laterally for a respective lobe length relative to the central longitudinal axis.

27. A brace according to claim 26 wherein the lobe portions of each said pair extend equidistantly of said central portion.

28. A brace according to claim 26 wherein said support piece is formed as a unitary one-piece construction of the stiff yet bendable material, said lobe portions being formed by notches located between adjacent ones of said lobe portions.

29. A brace according to claim 26 including at least three pairs of lobe portions, there being a primary pair of lobe portions and two secondary lobe portions, one on either side of said primary pair of lobe portions.

30. A brace according to claim 28 wherein said primary lobe portion is wider than said secondary lobe portions.

31. A brace according to claim 28 wherein the lobe portions of said primary pair extend a lateral distance away from said central portion that is greater than the lobe portions of said secondary pairs.

32. A brace according to claim 26 wherein said stiff yet bendable material is selected from a group consisting of: composite materials, plastic, aramid compounds, Kevlar®, graphite, plastic, ballistic-type materials and resin impregnated fabrics.
